(19) United States
(12) Patent Application Publication
(10) Pub. No.: US 2023/0049360 A1
(43) Pub. Date: Feb. 16, 2023

(54) METHOD FOR ASSESSING PITCHERS

(71) Applicant: ProPlayAI Inc., Oakville (CA)

(72) Inventors: Reed HANOUN, Oakville (CA); Mike SONNE, Oakville (CA)

(21) Appl. No.: 17/862,213

(22) Filed: Jul. 11, 2022

Related U.S. Application Data
(63) Continuation-in-part of application No. 16/843,432, filed on Apr. 8, 2020, now abandoned.
(60) Provisional application No. 62/830,801, filed on Apr. 8, 2019.
(60) Provisional application No. 62/983,995, filed on Mar. 2, 2020.

(51) Int. Cl. A63B 24/00; A61B 5/00; A61B 5/11

(52) U.S. Cl. CPC A63B 24/0006; A61B 5/0077; A61B 5/1116; A61B 5/1121; A61B 5/1128; A63B 2230/62; A63B 2220/30; A61B 2503/10

(57) ABSTRACT

The skeletal position of a pitcher at the moment of foot strike in the delivery of a pitch is compared against a plurality of skeletal positions, the plurality of skeletal positions being associated with a plurality of pitchers, each skeletal position being associated with the moment of foot strike in the delivery of a pitch. A value representative of a position of the pitch on a spectrum is provided, the spectrum being defined by a characteristic known about each of the pitchers of the plurality, the characteristic being Tommy John surgery or maximum fastball speed.

### CROSS-REFERENCE TO RELATED INVENTIONS

This is a continuation-in-part of U.S. patent application Ser. No. 16/843,432, filed Apr. 8, 2020, which claims priority to U.S. Provisional Patent Application Ser. No. 62/830,801, filed Apr. 8, 2019, and U.S. Provisional Patent Application Ser. No. 62/983,995, filed Mar. 2, 2020, all incorporated herein by reference.

### FIELD

The invention relates to the field of sports medicine.

### BACKGROUND

It is well known that the ulnar collateral ligament (UCL) can become stretched, frayed or torn through the repetitive stress of the throwing motion. As such, this is a commonplace baseball injury. Preventing injuries of this type in major league baseball would be highly desirable.

### SUMMARY OF THE INVENTION

Forming one aspect of the invention is a method comprising the steps:
comparing the skeletal position of a pitcher at the moment of foot strike in the delivery of the pitch against a plurality of skeletal positions,
   the plurality of skeletal positions being associated with a plurality of pitchers,
   each skeletal position being associated with the moment of foot strike in the delivery of a pitch;
providing a value representative of a position of the pitch on a spectrum,
wherein the spectrum is defined by a characteristic known about each of the pitchers of the plurality of pitchers.

According to another aspect, the characteristic can be maximum fastball velocity.

According to another aspect, the characteristic can be UCL injury resulting in ulnar collateral ligament reconstruction

According to another aspect,
the plurality of pitchers can include a plurality of pitchers that have undergone Tommy John surgery and a plurality of pitchers that have never undergone Tommy John surgery;
the skeletal positions associated with the pitchers that have undergone Tommy John surgery are associated with pitches delivered prior to the Tommy John surgery; and
the value is representative of a position of the pitch on a spectrum of pitch health wherein one end is defined by the skeletal positions of the pitchers that have never undergone Tommy John surgery and the other end is defined by the skeletal positions of the pitchers that have undergone Tommy John surgery.

According to another aspect, the skeletal positions are compared with reference to the parameters: elbow flexion; shoulder abduction; should external rotation; horizontal abduction; humeral rotation; forearm pronation; torso rotation; pelvis rotation; trunk tilt; lead knee flexion; lead foot rotation; and foot strike.

According to another aspect, the parameters can be indexed into bins as shown:

| Parameters | Bin 1 | Bin 2 | Bin 3 | Bin 4 | Bin 5 |
|---|---|---|---|---|---|
| Elbow Flexion | 55°> | 55-74° | 74-11° | 111-130° | 130°< |
| Shoulder Abduction | 83°> | 83-102° | 102°< | | |
| Shoulder external | 11°> | 11-67° | 67°< | | |
| rotation | | | | | |
| Horizontal abduction | −35°> | (−35)-(−13)° | −13°< | | |
| Humeral rotation | Inverted W | Flat Arm | Arm up | | |
| | (0-60°) | (60-120°) | (120-180°) | | |
| Forearm Pronation | Supinated | Neutral | Pronated | | |
| | (0-60°) | (60-120°) | (120-180°) | | |
| Torso Rotation | −36°> | (−36)-(−8)° | −8°< | | |
| Pelvis Rotation | 23°> | 23-49° | 49°< | | |
| Trunk Tilt | −5°> | (−5)-8° | 8°< | | |
| Lead Knee Flexion | 123°> | 123-143° | 143°< | | |
| Lead Foot Rotation | Closed | Angled | Home | Open | |
| Foot Strike | Heel up | Flat | Toe Up | | |

According to another aspect, the skeletal position can be defined by joint centres.

According to another aspect, the joint centres can be derived from 2D video imagery.

Advantages, features and characteristics of the present invention will become apparent upon review of the following detailed description, with reference to the appended drawings, latter being described hereinafter.

### DETAILED DESCRIPTION OF A NON-LIMITING EXAMPLE OF A METHOD EMBODYING THE INVENTION

As an initial matter, it should be understood that in this method, recourse is had to skeletal positions defined with reference to the characteristics indicated below

| Parameters | Bin 1 | Bin 2 | Bin 3 | Bin 4 | Bin 5 |
|---|---|---|---|---|---|
| Elbow Flexion | 55°> | 55-74° | 74-11° | 111-130° | 130°< |
| Shoulder Abduction | 83°> | 83-102° | 102°< | | |
| Shoulder external | 11°> | 11-67° | 67°< | | |
| rotation | | | | | |
| Horizontal abduction | −35°> | (−35)-(−13)° | −13°< | | |
| Humeral rotation | Inverted W | Flat Arm | Arm up | | |
| | (0-60°) | (60-120°) | (120-180°) | | |
| Forearm Pronation | Supinated | Neutral | Pronated | | |
| | (0-60°) | (60-120°) | (120-180°) | | |
| Torso Rotation | −36°> | (−36)-(−8)° | −8°< | | |
| Pelvis Rotation | 23°> | 23-49° | 49°< | | |
| Trunk Tilt | −5°> | (−5)-8° | 8°< | | |
| Lead Knee Flexion | 123°> | 123-143° | 143°< | | |
| Lead Foot Rotation | Closed | Angled | Home | Open | |
| Foot Strike | Heel up | Flat | Toe Up | | |

Persons of ordinary skill in body mechanics can routinely classify a known skeletal position in this manner.

Such skeletal positions can be developed by joint centres derived from 2D video imagery. Technologies suitable for this include WrnchAl. 3D marker-based motion capture systems could alternatively be used.

Against this backdrop, the method will be understood to comprise a comparing step, a providing step and a matching step.

In the comparing step, the skeletal position of a pitcher at the moment of foot strike in the delivery of the pitch, characterized as per the above, is compared against a plurality of skeletal positions similarly characterized, each skeletal position being associated with the moment of foot strike in the delivery of a pitch:
i. in one such comparison, the plurality of skeletal positions being associated with a plurality of pitchers, the plurality of pitchers including a plurality of pitchers that have undergone ulnar collateral ligament reconstruction (Tommy John surgery) and a plurality of pitchers that have never undergone Tommy John surgery, the skeletal positions associated with the pitchers that have undergone Tommy John surgery being indexed accordingly and associated with pitches delivered prior to the Tommy John surgery; and
ii. in another such comparison, the plurality of skeletal positions being associated with a plurality of pitchers, the skeletal positions being indexed according to maximum fastball speed.
   For greater certainty, the moment of front foot strike is the moment a pitcher's front foot makes contact with the ground and is the starting point of energy transfer up the kinetic chain

In the providing step, values are provided, each value being representative of a position of the pitch on a spectrum, wherein the spectrum is defined by a characteristic known about each of the pitchers of the plurality:
i. one of the values provided is representative of a position of the pitch on a spectrum of pitch health wherein one end is defined by the skeletal positions of the pitchers that have never undergone Tommy John surgery and the other end is defined by the skeletal positions of the pitchers that have undergone Tommy John surgery; and
ii. another of the values provided is representative of the position of the pitch on a spectrum of maximum fastball speed.

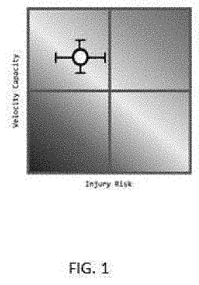

A graphical representation of such value provision is shown in FIG. 1. In this example, the datum is in the top left quadrant: this pitcher will be understood to have high velocity mechanics and high injury risk mechanics.

In the matching step, the skeletal position is compared against the skeletal positions and the pitcher having biomechanics most similar to that of the pitch is identified based upon a similarity score

Similarity Score=n=1nj=1j(pc−pa)2j

where

pc is the pitcher comparison (professional pitcher)

pa is the pitcher in the app

n is equal to the number of pitchers

j is equal to the number of joints and comparisons in question (segment length changes, joint angles, joint angle change speed, etc).

Persons of ordinary skill will readily appreciate that the foregoing has significant advantage, including but not limited to:
pitchers can modify delivery to decrease the likelihood of injury and increase maximum fastball speed;
coaches can give pitchers with poor mechanics more rest
coaches can remove pitchers from play when body mechanics appear to be slipping;
general managers can choose/pay players based upon their likely success and longevity
players can modify their technique to emulate other players

### Variations

Whereas a specific method is described, variations are of course possible.

For example, skeletal structures could be characterized otherwise than by reference to joint centres.

Other parameters could be used, as could different bins.

The values could be provided otherwise than via the graphic.

The skeletal structure could be examined at a different point in the pitch.

Whereas the description, for simplicity, speaks only to a single comparison being done, it will be appreciated that accuracy and reliability will improve based upon averaging, that is, the body mechanics of a pitcher will vary from pitch-to-pitch and more robust predictions about any given pitcher will be made if a plurality of pitches are compared and results averaged.

Accordingly, the invention should be understood to be limited only by the accompanying claims, purposively construed.

1. A method comprising the steps:
comparing the skeletal position of a pitcher at the moment of foot strike in the delivery of the pitch against a plurality of skeletal positions,
the plurality of skeletal positions being associated with a plurality of pitchers,
each skeletal position being associated with the moment of foot strike in the delivery of a pitch; and
providing a value representative of a position of the pitch on a spectrum,
wherein the spectrum is defined by a characteristic known about each of the pitchers of the plurality.

2. A method according to claim 1, wherein the characteristic is maximum fastball velocity.

3. A method according to claim 1, wherein the characteristic is UCL injury resulting in ulnar collateral ligament reconstruction.

4. A method according to claim 3, wherein
the plurality of pitchers includes a plurality of pitchers that have undergone ulnar collateral ligament reconstruction and a plurality of pitchers that have never undergone ulnar collateral ligament reconstruction;
the skeletal positions associated with the pitchers that have undergone ulnar collateral ligament reconstruction is associated with pitches delivered prior to the ulnar collateral ligament reconstruction; and
the value is representative of a position of the pitch on a spectrum of pitch health wherein one end is defined by the skeletal positions of the pitchers that have never undergone ulnar collateral ligament reconstruction and the other end is defined by the skeletal positions of the pitchers that have undergone ulnar collateral ligament reconstruction.

5. A method according to claim 1, wherein the skeletal positions are compared with reference to the parameters
elbow flexion;
shoulder abduction;
should external rotation;
horizontal abduction;
humeral rotation;
forearm pronation;
torso rotation;
pelvis rotation;
trunk tilt;
lead knee flexion;
lead foot rotation; and
foot strike.

6. A method according to claim 5, wherein for computational simplicity, the parameters are indexed into discrete bins:

| Parameters | Bin 1 | Bin 2 | Bin 3 | Bin 4 | Bin 5 |
|---|---|---|---|---|---|
| Elbow Flexion | 55°> | 55-74° | 74-11° | 111-130° | 130°< |
| Shoulder Abduction | 83°> | 83-102° | 102°< | | |
| Shoulder external | 11°> | 11-67° | 67°< | | |
| rotation | | | | | |
| Horizontal abduction | −35°> | (−35)-(−13)° | −13°< | | |
| Humeral rotation | Inverted W | Flat Arm | Arm up | | |
| | (0-60°) | (60-120°) | (120-180°) | | |
| Forearm Pronation | Supinated | Neutral | Pronated | | |
| | (0-60°) | (60-120°) | (120-180°) | | |
| Torso Rotation | −36°> | (−36)-(−8)° | −8°< | | |
| Pelvis Rotation | 23°> | 23-49° | 49°< | | |
| Trunk Tilt | −5°> | (−5)-8° | 8°< | | |
| Lead Knee Flexion | 123°> | 123-143° | 143°< | | |
| Lead Foot Rotation | Closed | Angled | Home | Open | |
| Foot Strike | Heel up | Flat | Toe Up | | |

7. A method according to claim 1, wherein the skeletal position is defined by joint centres.

8. A method according to claim 7, wherein the joint centres are derived from 2D video imagery.
